(19) United States
(12) Patent Application Publication
(10) Pub. No.: US 2024/0390396 A1
(43) Pub. Date: Nov. 28, 2024

(54) COMBINATIONS OF CANNABIS AND PLANT-DERIVED COMPOUNDS AS ANTI-CELL PROLIFERATION-RELATED DISEASE AGENTS

(71) Applicant: ARIEL SCIENTIFIC INNOVATIONS LTD., Ariel (IL)

(72) Inventors: Noa DAVIS, Petah Tikvah (IL); Igor KOMAN, Tel Aviv (IL); Natalya M. KOGAN, Kfar Adummim (IL)

(21) Appl. No.: 18/687,448

(22) PCT Filed: Aug. 29, 2022
(86) PCT No.: PCT/IL2022/050944

§ 371 Date: Feb. 28, 2024

Related U.S. Application Data
(60) Provisional application No. 63/238,171, filed on Aug. 29, 2021.

(51) Int. Cl. A61K 31/00; A61K 31/045; A61K 31/05; A61K 31/125; A61K 31/165; A61K 31/365; A61K 31/55; A61P 35/00

(52) U.S. Cl. CPC A61K 31/658; A61K 31/045; A61K 31/05; A61K 31/125; A61K 31/165; A61K 31/365; A61K 31/55; A61P 35/00

(57) ABSTRACT

The present invention discloses a pharmaceutical composition including a cannabinoid and a transient receptor potential cation channel (TRP) ligand, and methods of using same, such as for treating a subject afflicted with a cell proliferation related disease, or alleviating a symptom associated therewith.

### CROSS-REFERENCE TO RELATED APPLICATIONS

This application claims the benefit of priority of U.S. Provisional Patent Application No. 63/238,171, titled “COMBINATIONS OF CANNABIS AND PLANT-DERIVED COMPOUNDS AS ANTI-CANCER AGENTS”, filed 29 Aug. 2021, the contents of which are incorporated herein by reference in their entirety.

### FIELD OF INVENTION

The present invention relates to combination of a cannabinoid and transient receptor potential cation channel TRP) ligands, and methods of using same, such as for treating a cell proliferation related disease, or alleviating a symptom associated therewith.

### BACKGROUND

Cannabinoids are used therapeutically by many cancer patients owing to their analgesic, anti-emetic and appetite stimulant properties. However, a long line of preclinical evidence suggests that cannabinoids may be useful as anti-cancer agents as well. In vitro studies show that cannabinoids and cannabinoid-like compounds inhibit proliferation and cell migration in different breast carcinoma cell lines (e.g., MCF-7, EFM-19, T-47D, MDA-MB-231, MDA-MB-468, MDA-MB-436, 4T1, TSA-E1, EVSA-T, SkBr3, HTB-126, etc.). Other studies have shown that cannabinoids can block cell cycle progression and cell growth and induce programmed cell death by inhibiting pro-oncogenic signaling pathways, such as the extracellular-signal-regulated kinase pathway. Importantly, these therapeutic effects are dose-dependent, and require extremely high dosage of cannabinoids. Moreover, usage of low dose cannabinoids on cancerous cell lines is either non-efficient or pro-tumorigenic (personal observation). Despite their proven effect in vitro, cannabinoids are poorly soluble in water, and subjected to extensive first pass metabolism in the gastrointestinal tract, thereby leading to a limited oral bioavailability, which hampers their effectiveness in vivo.

Phytocannabinoids exert their function through binding to the receptors of the endocannabinoid system, including, e.g., CB1, CB2, TRPV1, PPARs, GPR18, GPR55, GPR119, and others. Taking into consideration the poor bioavailability of phytocannabinoids, many attempts to use synthetic, more potent ligands of those receptors, were made. Nevertheless, as the endocannabinoid system is involved in many physiological processes, such as temperature sensation, hunger and mood stabilization, etc., the resultant drugs either failed at the clinical trials due to safety issues or removed from the market owing to serious adverse effects.

Inflammation contributes to the pathogenesis of many diseases. Chronic inflammation can lead to cardiovascular diseases, gastrointestinal diseases, obesity, asthma, arthritis, neurodegenerative diseases, cancer and more.

Currently, the drugs used to treat inflammation are most often non-steroidal anti-inflammatory drugs (NSAIDs) and steroids. Monoclonal antibodies to cytokines, tumor necrosis factor (TNF), interleukin (IL)6 and IL12/23 are used if these do not work. NSAIDs work by inhibiting the activity of cyclooxygenase enzymes (COX-1 or COX-2). In cells, these enzymes are involved in the synthesis of prostaglandins, which are associated with inflammation, and thromboxanes, which are involved in blood clotting. Most NSAIDs are non-selective and inhibit the activity of both COX-1 and COX-2. These NSAIDs, while reducing inflammation, also inhibit platelet aggregation and increase the risk of gastrointestinal ulcers/bleeds. Side effects can include an increased risk of gastrointestinal ulcers and bleeds, heart attack, and kidney disease. COX-2 selective inhibitors have fewer gastrointestinal side effects but promote thrombosis and some of these agents substantially increase the risk of heart attack. By inhibiting physiological COX activity, all NSAIDs increase the risk of kidney disease and through a related mechanism, heart attack. In addition, NSAIDs can blunt the production of erythropoietin resulting in anemia, since hemoglobin synthesis depends on this hormone.

Of equal if not greater concern are side effects associated with long-term use of steroidal drugs. Although highly effective in the elimination of inflammation, they can cause obesity, growth retardation in children, and even lead to convulsions and psychiatric disturbances, osteoporosis, adrenal suppression, hyperglycemia, dyslipidemia, cardiovascular disease, Cushing's syndrome, immunosuppression, an increase in the rate of infections.

In the current disclosure, the inventors show that the anti-cell proliferation activity of cannabinoids, e.g., cannabidiol (CBD), and tetrahydrocannabinol (THC), used as either a pure material or as a component of Cannabis sativa extract, can be augmented by molecules that interact with a TRP, e.g., a TRPV1 receptor, or a TRPA1 receptor.

There is still a great need for pharmaceutical compositions comprising a cannabinoid, e.g., CBD, or THC, having increased anti-cell proliferation activity in vivo.

### SUMMARY

According to some embodiments, the present invention is based, in part, on the findings that the anti-cancer and anti-inflammatory activity attributed to cannabinoid, e.g., CBD, and THC, either used as a pure material or as a component of Cannabis sativa extract, was synergistically enhanced when combined with molecules that interact with a transient receptor potential cation channel receptor (TRP), namely, subfamily V member 1 TRP (TRPV1), or subfamily A member 1 TRP (TRPA1).

According to one aspect, there is provided a pharmaceutical composition comprising a cannabinoid and a transient receptor potential cation channel (TRP) ligand, for use in treatment of a cell proliferation related disease, or alleviation of a symptom associated therewith, in a subject in need thereof.

According to another aspect, there is provided a combination of a cannabinoid and a TRP ligand, for use in the treatment of a cell proliferation related disease, in a subject in need thereof.

According to another aspect, there is provided a method for increasing or enhancing an activity of a cannabinoid in a subject in need thereof, comprising administering to the subject a pharmaceutical composition comprising a TRP ligand, wherein said activity is selected from the group consisting of: anti cell proliferation, anti-inflammation, analgesic, and any combination thereof.

According to another aspect, there is provided a method for increasing or enhancing an activity of a TRP ligand in a subject in need thereof, comprising administering to the subject a pharmaceutical composition comprising a cannabinoid, wherein said activity is selected from the group consisting of: anti cell proliferation, anti-inflammation, analgesic, and any combination thereof.

According to another aspect, there is provided a method for ameliorating or treating a subject afflicted with a cell proliferation related disease, or a symptom associated therewith, the method comprising administering to the subject a therapeutically effective amount of a pharmaceutical composition comprising a cannabinoid and a TRP ligand, thereby ameliorating or treating the subject afflicted with the cell proliferation related disease.

In some embodiments, the cannabinoid and the TRP ligand are present in the composition at a molar ratio ranging from 1:1 (M:M) to 1:1,000 (M:M).

In some embodiments, the cannabinoid is selected from cannabidiol (CBD) or tetrahydrocannabinol (THC).

In some embodiments, the TRP is selected from a subfamily V member 1 TRP (TRPV1) or a subfamily A member 1 TRP (TRPA1).

In some embodiments, the TRPV1 ligand is selected from the group consisting of: capsazepine, capsaicin, thapsigargin, and any combination thereof.

In some embodiments, the TRPA1 ligand is selected from the group consisting of: menthol, camphor, carvacrol, and any combination thereof.

In some embodiments, the cannabinoid is present as a highly purified extract of Cannabis.

In some embodiments, the cannabinoid is synthetically produced.

In some embodiments, the pharmaceutical composition further comprises a pharmaceutically acceptable carrier.

In some embodiments, the cell proliferation related disease is inflammation or cancer.

In some embodiments, cancer is epithelial cancer.

In some embodiments, epithelial cancer is cancer in a tissue selected from the group consisting of: breast, lung, pancreas, colon, and any combination thereof.

In some embodiments, breast cancer comprises triple negative metastatic adenocarcinoma.

In some embodiments, the symptom associated with the cell proliferation related disease comprises pain.

In some embodiments, the cannabinoid is formulated within a first pharmaceutical composition and said TRP ligand is formulated within a second pharmaceutical composition.

In some embodiments, the administering is in a synergistically effective amount.

In some embodiments, the administered cannabinoid and TRP ligand are at a molar ratio ranging from 1:1 (M:M) to 1:1,000 (M:M) in the pharmaceutical composition.

In some embodiments, the treating comprises: reducing the rate of cell proliferation, reducing the number of proliferating cells, reducing the survival rate of proliferating cells, reducing the levels of tumor necrosis factor alpha (TNFα), reducing pain associated with said cell proliferation related disease, or any combination thereof, in the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1D include graphs showing that cannabidiol (CBD) and cannabis reduce cell survival of MDA-MB-231 cells. (1A) Twenty four (24) hr treatment of MDA-MB-231 breast cancer cells with CBD-enriched cannabis induced dose-dependent reduction in cell viability, presented as percentage of the optical density (OD) values of cells treated with vehicle (DMSO). Data are expressed as mean±SD. Each experiment was performed at least 3 times. (1B) Twenty-four (24) hr treatment of the same cells with either CBD-enriched cannabis or antagonists of the extracellular surface (ECS) receptors (see Table 1 for details) or their combinations have shown that the cytotoxic effect of cannabis (seen in the first bar and further represented by a dashed line) is further enhanced by the antagonist of the TRPV1 receptor, capsazepine (p. value <0.00001). Data are expressed as mean±SD. Each experiment was performed at least 3 times. (1C) Dose response analysis showed that the efficacy of the combination of cannabis (Cann) and capsazepine (CAP) was always superior to that of cannabis alone (p. value <0.0005), and superior to that of capsazepine at the 2 highest doses tested (p. value <0.005 and 0.05, respectively). Data are expressed as mean±SD. Each experiment was performed at least 6 times. (1D) Twenty-four (24) hr treatment with 2.5 μM CBD and 32 μM capsazepine (CAP) significantly enhanced the cytotoxic effect of CBD, p. value <0.005. Data are expressed as box and whisker plot, which displays the minimum, maximum, sample median, and the 25-75% quartiles. Each experiment was performed at least 6 times.

FIG. 2 includes a vertical bar graph showing that capsazepine enhances the cytotoxic effect of cannabis in breast cancer cell lines. Twenty-four (24) hr treatment of MCF7 and T47D breast cancer cells with a combination of cannabis extract (cann) containing 4 μM CBD and 32 μM capsazepine (CAP) had a cytotoxic effect that was higher than that of cannabis (for both cell type, p. value <0.005), and higher in comparison to capsazepine for MCF7 cells (p. value <0.0005). Exposure of MDA-MB-468 cells to cannabis killed most of the cells, and addition of capsazepine had a small contribution to the cytotoxicity. Exposure of the cells to 1:2 dilution of the materials showed the same potentiation of the combined effect in comparison to each of the materials alone (p. value <0.005). Data are expressed as mean±SD. Each experiment was performed at least 6 times.

FIGS. 3A-3D include vertical bar graphs showing that capsaicin and thapsigargin enhance the cytotoxic effect of cannabis in breast cancer cell lines. (3A) Twenty-four (24 hr) treatment of MDA-MB-231 with cannabis extract (cann) containing various concentrations of CBD (0.5-4 μM), various concentrations of capsaicin (50-400 μM) or combination thereof showed that the combined effect was significantly stronger than that cannabis, especially at high doses (p. value ranges between 10 e⁻⁸ and 0.04). The effect of the combination was stronger than that of capsaicin alone at the highest dose tested (p. value <0.00005). (3B) Twenty-four (24 hr) treatment of MCF7, MDA-MB-231 and T47D cells with cannabis extract (cann) containing 4 μM CBD, 400 μM capsaicin or combination thereof showed that the combined effect was significantly stronger than that of each of the individual materials (p. value <0.005). Similar findings were found in MDA-MB-468 cells treated with 1:2 diluted materials. Data are expressed as mean±SD. Each experiment was performed at least 6 times. (3C) Twenty-four (24 hr) treatment of MDA-MB-231 with cannabis extract (cann) containing various concentrations of CBD (0.5-4 μM), various concentrations of thapsigargin (THAPS, 0.63-5 μM) or combination thereof showed that the combined effect was significantly stronger than that cannabis for all doses tested (p. value ranges between 10e-8 and 10e-6). The effect of the combination was stronger than that of thapsigargin alone at the three highest doses tested (p. value ranges between 10 e⁶ and 0.04). (3D) Twenty-four (24 hr) treatment of MCF7, MDA-MB-231 and T47D cells with cannabis extract (cann) containing 4 μM CBD, 5 μM thapsigargin or combination thereof showed that the combined effect was significantly stronger than that of each of the individual materials (p. value <0.005). Similar findings were found in MDA-MB-468 cells treated with 1:2 diluted materials. Data are expressed as mean±SD. Each experiment was performed at least 6 times.

FIG. 4 includes a vertical bar graph showing that the effect of cannabis/capsaicin and cannabis/thapsigargin on various cancer cell lines. Twenty-four (24) hr treatment of H1975, Panc1, BxPc3, Hs578T, and LS174t with cannabis (cann) containing 4 μM CBD, with or without 400 μM capsaicin showed that the cannabis/capsaicin combination was more cytotoxic then cannabis alone in Panc1, Hs578T and LS174t cells. A similar experiment performed with cannabis containing 4 μM CBD, with or without 5 μM thapsigargin showed that the cannabis/thapsigargin combination was more cytotoxic then cannabis alone in all cell types tested (p. value <0.005). Data are expressed as mean±SD. Each experiment was done at least 3 times.

FIGS. 5A-5B include graphs showing the effect of CBD on nitric oxide (NO) secretion by macrophages. (5A) Twenty-four (24 hr) treatment of RAW 264.7 macrophage cell line with LPS and increasing doses of CBD (0.63-5 μM) have demonstrated that CBD reduced the secretion of NO in a dose dependent manner. Data are presented as percentage of the values measured for cells treated with LPS and vehicle (DMSO). Data are expressed as mean±SD. Each experiment was performed at least 3 times. (5B) Twenty-four (24 hr) treatment of the same cells with LPS, CBD, antagonists of the ECS receptors (see table 2 for details) or their combinations have shown that 2.5 μM CBD (seen in the first bar and further represented by a dashed line) reduced NO secretion to 6±5% of the one measured for control cells. Capsazepine and GW6471 (TRPV1 and PPARα antagonists, respectively) had a mild effect on NO secretion, and the combination of 2.5 μM CBD and 10 μM capsazepine significantly reduced it to 2±3% of the control (p. value <0.05).

FIGS. 6A-6B include vertical bar graph showing a synergistic anti-cancer effect attributed to a combination of cannabinoid and a TRPV1 ligand, in vivo in a murine model. (6A) A graph showing tumors count in lungs. Mice infected with melanoma cells via tail vein (a model for metastatic tumors), were treated with CBD, Thapsigargin, or a combination thereof. The treatment started a week post tumor infection and continued for 3 weeks. (6B) A graph showing lungs weight of mice infected and treated as in 6A. In 6A-6B, data are expressed as mean±SEM. *p value <0.05; **p value <0.01; and ***p value <0.001.

FIGS. 7A-7C include vertical bar graph showing an analgesic and anti-inflammatory effect attributed to a combination of cannabinoid and a TRPA1 ligand, in vivo in a murine model. (7A-7B) Paw swelling of mice injected with zimozan into the paw and immediately treated with: (i) CBD; (ii) a TRPA1 ligand: menthol, camphor, or carvacrol; or (iii) combinations of (i) and (ii). Results reflect measurements conducted 6 hours post treatment (7A) 24 hours post treatment (7B), respectively. (7C) Pain test on the inflamed paw of 7A-7B was examined using von Frey filaments, and results are expressed as grams of pressure before paw withdrawal. Data are expressed as mean±SEM. *p value <0.05; **p value <0.01; and ***p value <0.001.

FIGS. 8A-8C include vertical bar graphs. (8A) Paw swelling of mice injected with zimozan into the paw and immediately treated by THC, menthol or both. Data are expressed as mean±SEM. *—p. value <0.05 **—p. value <0.01, ***—p. value <0.001. (8B) Pain test on the inflamed paw by von Frei filaments, expressed as grams of pressure before paw withdrawal. Data are expressed as mean±SEM. *—p. value <0.05 **—p. value <0.01, ***—p. value <0.001. (8C) TNFα plasma levels 24 h after zymosan and the treatment injections. Data are expressed as mean±SEM. *—p. value <0.05 **—p. value <0.01, ***—p. value <0.001.

### DETAILED DESCRIPTION

The present invention provides compositions comprising a cannabinoid or a functional analog thereof, pure or as apart of cannabis extract, and a TRP ligand, or a plurality thereof, or any combination thereof, and methods of using same, such as for treating or ameliorating a cell proliferation related disease, or a symptom associated therewith, in a subject in need thereof.

The present invention provides compositions comprising a cannabinoid or a functional analog thereof, pure or as apart of cannabis extract, and a TRP ligand, or a plurality thereof, or any combination thereof, and methods of using same, such as for treating pain, in a subject in need thereof.

In some embodiments, there is provided a composition comprising a cannabinoid or a functional analog thereof, pure or as apart of cannabis extract, and a TRP ligand, or a plurality thereof, or any combination thereof, and methods of using same, such as for treating or ameliorating a cell proliferation related disease, in a subject in need thereof.

### Cannabinoids and TRP Binding Molecules

According to some embodiments, there is provided a composition comprising a cannabinoid and a transient receptor potential cation channel (TRP) antagonist.

In some embodiments, the composition is a synergistic composition.

According to some embodiments, there is provided a synergistic composition comprising a cannabinoid and a transient receptor potential cation channel (TRP) antagonist.

According to some embodiments, there is provided a composition comprising cannabidiol (CBD) and a transient receptor potential cation channel subfamily V member 1 (TRPV1) antagonist.

According to some embodiments, there is provided a composition comprising cannabidiol (CBD) and a transient receptor potential cation channel subfamily V member 1 (TRPA1) antagonist.

According to some embodiments, there is provided a composition comprising tetrahydrocannabinol (THC) and a transient receptor potential cation channel subfamily V member 1 (TRPV1) antagonist.

According to some embodiments, there is provided a composition comprising tetrahydrocannabinol (THC) and a transient receptor potential cation channel subfamily V member 1 (TRPA1) antagonist.

In some embodiments, a cannabinoid is a phytocannabinoid.

As used herein, the term “phytocannabinoid” refers to a cannabinoid that originates in nature from a Cannabis plant, or to whole Cannabis extract rich in a cannabinoid, such as, but not limited to CBD-rich extract). Examples of cannabinoids include, but are not limited to, cannabidiol (CBD), cannabidivarin (CBDV), (−)-Δ⁹-trans-tetrahydrocannabinol (Δ⁹-THC), (−)-Δ⁹-trans-tetrahydrocannabinolic acid (Δ⁹-THCA), (−)-Δ⁹-trans-tetrahydrocannabivarin (Δ⁹-THCV), (−)-Δ⁹-trans-tetrahydrocannabivarinic acid (Δ⁹-THCVA), cannabinol (CBN), cannabivarin (CBNV), cannabicyclol (CBL), cannabigerol (CBG), cannabigerovarin (CBGV), cannabidiolic acid (CBDA), cannabichromene (CBC), cannabichromenic acid (CBCA) and any derivative thereof.

In some embodiments, the cannabinoid comprises or is CBD.

In some embodiments, the cannabinoid comprises or is THC.

In some embodiments, the cannabinoid comprises or is selected from CBD and THC.

In some embodiments, the composition comprises CBD and at least one TRPV1 antagonist selected from: capsazepine, capsaicin, thapsigargin, or any combination thereof.

In some embodiments, the composition comprises CBD and capsazepine.

In some embodiments, the composition comprises CBD and capsaicin.

In some embodiments, the composition comprises CBD and thapsigargin.

In some embodiments, the composition comprises CBD and at least one TRPA1 antagonist selected from: menthol, camphor, carvacrol, or any combination thereof.

In some embodiments, the composition comprises CBD and at least one TRPA1 antagonist selected from: menthol, camphor, carvacrol, or any combination thereof.

In some embodiments, the composition comprises CBD and menthol.

In some embodiments, the composition comprises CBD and camphor.

In some embodiments, the composition comprises CBD and carvacrol.

In some embodiments, the composition comprises THC and at least one TRPV1 antagonist selected from: capsazepine, capsaicin, thapsigargin, or any combination thereof.

In some embodiments, the composition comprises THC and capsazepine.

In some embodiments, the composition comprises THC and capsaicin.

In some embodiments, the composition comprises THC and thapsigargin.

In some embodiments, the composition comprises THC and at least one TRPA1 antagonist selected from: menthol, camphor, carvacrol, or any combination thereof.

In some embodiments, the composition comprises THC and menthol.

In some embodiments, the composition comprises THC and camphor.

In some embodiments, the composition comprises THC and carvacrol.

As used herein, the terms “transient receptor potential cation channel” or “TRP” binding molecules encompass any compound or agent capable of binding to a TRP protein.

In some embodiments, there is provided a TRP ligand.

In some embodiments, the TRP ligand is a competitive inhibitor. In some embodiments, the TRP ligand is a non-competitive inhibitor. In some embodiments, the TRPV1 ligand is selected from: a small molecule, a polypeptide, or an antibody.

In some embodiments, the TRP ligand is an antagonist. In some embodiments, the TRP ligand is an agonist.

In some embodiments, the TRP ligand comprises any molecule which binds to TRP and inhibits the signal transduction signaling of TRP. In some embodiments, the inhibition is immediate or rapidly occurring. In some embodiments, the inhibition is induced by TRP desensitization due to the binding of the ligand, as disclosed herein.

In some embodiments, the TRP ligand is a synthetic compound. In some embodiments, the TRP ligand is obtained, derived, or extracted from a plant, including any cell, tissue, organ, portion, fraction, derived therefrom, or any combination thereof.

In some embodiments, the TRP comprises a superfamily V member 1 TRP (TRPV1).

In some embodiments, the TRP comprises a superfamily A member 1 TRP (TRPA1).

In some embodiments, a TRPV1 ligand is selected from: capsazepine, capsaicin, thapsigargin, or any combination thereof.

In some embodiments, a TRPA1 ligand is selected from: menthol, camphor, carvacrol, or any combination thereof.

In some embodiments, the cannabinoid and TRP antagonist or ligand are present in the composition at a molar ratio ranging from 1:1 (M:M) to 1:200 (M:M), 1:2 (M:M) to 1:150 (M:M), 1:3 (M:M) to 1:100 (M:M), 1:1 (M:M) to 1:200 (M:M), 1:2 (M:M) to 1:40 (M:M), 1:1 (M:M) to 1:100 (M:M), 1:1 (M:M) to 1:8 (M:M), 1:10 (M:M) to 1:1,000 (M:M), 1:5 (M:M) to 1:5,000 (M:M), or 1:1 (M:M) to 1:16 (M:M). Each possibility represents a separate embodiment of the invention.

In some embodiments, the cannabinoid is present as a highly purified extract of Cannabis.

In some embodiments, the cannabinoid is synthetically produced.

As used herein, the term “synthetic” refers to a compound, a cannabinoid, for example CBD, that is manufactured using chemical means rather than by a plant.

In some embodiments, a synthetic cannabinoid comprises a biosynthetic cannabinoid.

As used herein, the term “biosynthetic” refers to a cannabinoid being produced and/or extracted from a transgenic, transformed, transfected, or a recombinant cell.

In some embodiments, the composition is for use in treatment of a cell proliferation related disease, in a subject in need thereof.

According to some embodiments, there is provided a combination of a cannabinoid as disclosed herein and a TRP antagonist, for use in the treatment of a cell proliferation related disease, or a symptom associated therewith, in a subject in need thereof.

In some embodiments, the cannabinoid is formulated within a first composition and TRP antagonist or ligand is formulated within a second composition.

In some embodiments, a composition is a pharmaceutical composition.

In some embodiments, the composition further comprises a pharmaceutically acceptable carrier.

In some embodiments, the present invention is directed to a composition comprising a plant extract, including any portion or fraction derived therefrom. In some embodiments, a plant extract is derived from a plant comprising cannabinoids. In some embodiments, the plant extract of the invention is derived from a Cannabis plant. In some embodiments, a plant extract is derived from Cannabis sativa, Thapsia garganica, a plant belonging to the genus Capsicum, or any combination thereof.

In some embodiments, the cannabis plant comprises Cannabis indica.

In some embodiments, the composition comprises or consists of plant extracts including fractions, portions, compounds, isolates, or any combination thereof.

As used herein, the term “extract” comprises the whole extract, a fraction thereof, a portion thereof, an isolated compound therefrom, or any combination thereof.

In some embodiments, the extract is derived from a plant material.

In one embodiment, the Cannabis derived substances used in the composition and methods as described herein include a cannabinoid, such as, but not limited to CBD, or a functional analog, isomer, enantiomer, salt thereof, or any combination thereof. In one embodiment, the composition described herein comprises purified or substantially purified (e.g., greater than 80% w/w, 85% w/w, 90%, w/w 95% w/w or 97% w/w) cannabinoid. In some embodiments of the methods described herein, purified or substantially purified (e.g., greater than 80% w/w, 85% w/w, 90%, w/w 95% w/w or 97% w/w) cannabinoid, or a functional analog, isomer, enantiomer, salt thereof, or any combination thereof, is administered to a subject afflicted with a cell proliferation related disease.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

A pharmaceutically-acceptable carrier suitable for the preparation of unit dosage form of a composition as described herein for peroral administration is well-known in the art.

In some embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmellose sodium, crospovidone, guar gum, sodium starch glycolate), additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), stabilizers (e.g. hydroxypropyl cellulose, hydroxypropylmethyl cellulose), viscosity increasing agents (e.g. carbomer, colloidal silicon dioxide, ethyl cellulose, guar gum lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), polymer coatings (e.g., poloxamers or poloxamines), and/or coating and film forming agents (e.g. ethyl cellulose, acrylates, polymethacrylates).

In some embodiments, preparation of effective amount or dose can be estimated initially from in vitro assays. In one embodiment, a dose can be formulated in animal models, and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

### Method of Treatment

According to some embodiments, there is provided a method for increasing or enhancing the therapeutic efficacy of a cannabinoid in a subject in need thereof, comprising administering to the subject a pharmaceutical composition comprising TRP antagonist or a ligand.

According to some embodiments, there is provided a method for increasing or enhancing the therapeutic efficacy of a TRP antagonist or ligand in a subject in need thereof, comprising administering to the subject a pharmaceutical composition comprising a cannabinoid.

In some embodiments, the method comprises increasing or enhancing an activity of a cannabinoid in the subject. In some embodiments, the method comprises increasing or enhancing an activity of a TRP antagonist or ligand in the subject. In some embodiments, the activity is selected from: anti cell proliferation, anti-inflammation, analgesic, or any combination thereof.

According to some embodiments, there is provided a composition comprising a cannabinoid for use in increase or enhancement of an activity of a TRP antagonist or ligand, wherein the activity is selected from: anti cell proliferation, anti-inflammation, analgesic, or any combination thereof.

According to some embodiments, there is provided a composition comprising a TRP antagonist or ligand for use in increase or enhancement of an activity of a cannabinoid, wherein that activity is selected from: anti cell proliferation, anti-inflammation, analgesic, or any combination thereof.

According to some embodiments, there is provided a method for ameliorating or treating a subject afflicted with a cell proliferation related disease.

According to some embodiments, there is provided a method for treating a cell proliferation related disease in a subject in need thereof.

According to some embodiments, there is provided a method for treating cancer in a subject in need thereof.

According to some embodiments, there is provided a method for treating inflammation in a subject in need thereof.

According to some embodiments, there is provided a method for treating or reducing pain in a subject in need thereof.

In some embodiments, the method comprises increasing or enhancing the anti-cell proliferation activity of CBD, THC, TRPV1 antagonist or ligand, TRPA1 antagonist or ligand, or any combination thereof.

In some embodiments, the method comprises administering to the subject a therapeutically effective amount of a pharmaceutical composition comprising a cannabinoid and a TRP antagonist or ligand, thereby ameliorating or treating the subject afflicted with the cell proliferation related disease, or alleviating a symptom associated therewith.

In some embodiments, administering comprises providing a cannabinoid and TRP ligand in a therapeutically effective amount, synergistically effective amount, or both, to the subject.

As used herein, the term “synergistically effective amount” comprises any weight or concentration of a cannabinoid and TRP ligand, as long as their molar ratio ranges from: 1 (M:M) to 1:1,000 (M:M), 1:2 (M:M) to 1:500 (M:M), 1:3 (M:M) to 1:250 (M:M), 1:1 (M:M) to 1:200 (M:M), 1:2 (M:M) to 1:40 (M:M), 1:10 (M:M) to 1:600 (M:M), 1:1 (M:M) to 1:8 (M:M), or 1:1 (M:M) to 1:16 (M:M). Each possibility represents a separate embodiment of the invention.

In some embodiments, the term “synergistically effective amount” comprises any w:w or M:M ratio of a cannabinoid and TRP antagonist or ligand, wherein the therapeutic activity of the cannabinoid and TRP antagonist or ligand combined is greater than the summation of the individual therapeutic activities of the cannabinoid and TRP antagonist or ligand.

The present invention contemplates any synergistic composition comprising a cannabinoid and a TRP antagonist or ligand for use in the methods as disclosed herein.

In some embodiments, the subject is afflicted with a cell proliferation related disease.

In some embodiments, the subject is a mammal. In some embodiments, the subject is a human subject.

As used herein, the term “cell proliferation related disease” refers to any disease or condition associated therewith comprising or characterized by increased, enhanced, unregulated, dysregulated, abnormal, excessive, or any combination thereof, of cell proliferation.

In some embodiments, a cell proliferation related disease comprises inflammation, cancer, or both.

In some embodiments, cancer comprises an epithelial cancer.

As used herein, the terms “epithelial cancer” and “carcinoma” are interchangeable and refer to any malignancy that develops from or involves epithelial cells.

In some embodiments, carcinoma is selected from: adenocarcinoma, squamous cell carcinoma, adenosquamous carcinoma, anaplastic carcinoma, large cell carcinoma, small cell carcinoma, or any combination thereof.

As used herein, “cancer” encompasses diseases associated with cell proliferation.

Non-limiting types of cancer include carcinoma, adenocarcinoma, sarcoma, lymphoma, leukemia, blastoma and germ cells tumors. In one embodiment, carcinoma refers to tumors derived from epithelial cells including but not limited to breast cancer, prostate cancer, lung cancer, pancreas cancer, skin cancer, stomach, liver, and colon cancer. In one embodiment, sarcoma refers of tumors derived from mesenchymal cells including but not limited to sarcoma botryoides, chondrosarcoma, Ewing's sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to Hodgkin lymphoma, non-Hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refer to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, non-germinomatous or non-seminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

In some embodiments, cancer is in a tissue selected from: breast, lung, pancreas, colon, or any combination thereof.

In some embodiments, cancer comprises metastatic cancer.

In some embodiments, breast cancer comprises triple negative metastatic adenocarcinoma.

In some embodiments, treating comprises: reducing the rate of cell proliferation, reducing the number of proliferating cells, reducing the survival rate of proliferating cells, reducing the levels of tumor necrosis factor alpha (TNFα), reducing pain associated with the cell proliferation related disease, or any combination thereof, in the subject.

In some embodiments, TNFα levels comprise: mRNA levels, protein levels, or both. In some embodiments, TNFα levels comprise circulating protein levels (e.g., in the blood).

In some embodiments, there is provided a method for reducing pain associated with a cell proliferation related disease in a subject in need thereof, comprising administering a therapeutically effective amount of the composition disclosed herein.

As used herein, the terms “administering”, “administration”, and like terms refer to any method which, in sound medical practice, delivers a composition containing an active agent to a subject in such a manner as to provide a therapeutic effect. One aspect of the present subject matter provides for dermal or transdermal administration of a therapeutically effective amount of a composition of the present subject matter to a subject in need thereof. Other suitable routes of administration can include oral, inhalation, dermal, transdermal, parenteral, subcutaneous, intravenous, intramuscular, or intraperitoneal. In some embodiments, the administering is systemic administering. In some embodiments, the administering is to the wounded site.

Administering the composition to a specific site in the subject may be performed with any method known in the art. This may include with an applicator, in the form of a gel or cream, as well as on a scaffold, wrap or bandage.

As used herein, the terms “treatment” or “treating” of a disease, disorder or condition (e.g., a wound) encompasses alleviation of at least one symptom thereof, a reduction in the severity thereof, or inhibition of the progression thereof. Treatment need not mean that the disease, disorder or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, “treating” comprises ameliorating.

In some embodiments, reduce, reducing, inhibit, or inhibiting is at least 5%, 10%, 25%, 30%, 40%, 50%, 60%, 70%, 80%, 90%, 95%, or 100% reduction or inhibition compared to a control, or any value and range therebetween. Each possibility represents a separate embodiment of the invention.

In some embodiments, increase, increasing, enhance, or enhancing is at least 5%, 10%, 35%, 50%, 80%, 100%, 150%, 270%, 400%, 650%, 800%, or 1,000% increase compared to a control, or any value and range therebetween. Each possibility represents a separate embodiment of the invention.

In some embodiments, reduce, reducing, inhibit, or inhibiting is 5-100%, 10-100%, 25-100%, 30-100%, 40-100%, 50-100%, 60-100%, 70-100%, 80-100%, 90-100%, 95-100%, or 97-100% reduction or inhibition compared to a control. Each possibility represents a separate embodiment of the invention.

In some embodiments, increase, increasing, enhance, or enhancing is 5-100%, 10-200%, 35-400%, 50-500%, 80-550%, 100-600%, 150-700%, 270-750%, 400-850%, 650-900%, 800-1000%, or 1,000-1,200% increase compared to a control, or any value and range therebetween. Each possibility represents a separate embodiment of the invention.

In some embodiments, compositions for use in the methods of this invention comprise solutions or emulsions, which in some embodiments are aqueous solutions or emulsions comprising a safe and effective amount of the cannabinoid of the present invention (e.g., CBD) and other compounds as described herein, including excipients intended for topical intranasal administration.

In another embodiment, the composition is administered by intravenous, intra-arterial, or intramuscular injection of a liquid preparation. In some embodiments, liquid formulations include solutions, suspensions, dispersions, emulsions, oils and the like. In one embodiment, the composition is administered intravenously, and is thus formulated in a form suitable for intravenous administration. In another embodiment, the composition is administered intra-arterially, and is thus formulated in a form suitable for intra-arterial administration. In another embodiment, the composition is administered intramuscularly, and is thus formulated in a form suitable for intramuscular administration.

Further, in another embodiment, the composition is administered topically to body surfaces, and is thus formulated in a form suitable for topical administration. Suitable topical formulations include gels, ointments, creams, lotions, drops and the like. For topical administration, the active ingredients disclosed herein, e.g., one or more cannabinoids, are combined with an additional appropriate therapeutic agent or agents, prepared and applied as solutions, suspensions, or emulsions in a physiologically acceptable diluent with or without a pharmaceutical carrier.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, the composition is a suspension, a solution or an emulsion in oily or aqueous vehicle, and contains a suspending, a stabilizing and/or a dispersing agent.

In some embodiments, a composition for parenteral administration includes aqueous solution of the active preparation in water-soluble form. Additionally, suspensions of the active ingredients, in some embodiments, are prepared as appropriate oily or water-based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contains suitable stabilizers or agents which increase the solubility of the active ingredients to allow for the preparation of highly concentrated solutions.

In another embodiment, the composition delivered in a controlled release system is formulated for intravenous infusion, implantable osmotic pump, transdermal patch, liposomes, or other modes of administration. In one embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, further polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Compositions are formulated, in some embodiments, for atomization and inhalation administration. In another embodiment, compositions are contained in a container with attached atomizing means.

In one embodiment, the preparation of the present invention is formulated in rectal compositions such as suppositories or retention enemas, using, e.g., conventional suppository bases such as cocoa butter or other glycerides.

In one embodiment, the amount of a composition to be administered will be dependent on the subject being treated, the severity of the affliction, the manner of administration, the judgment of the prescribing physician, etc.

The dosage administered will be dependent upon the age, health, and weight of the recipient, kind of concurrent treatment, if any, frequency of treatment, and the nature of the effect desired.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials

All studies were approved by the Medical Cannabis Unit of the Ministry of health. Cannabis extract (Cat. #47TOP) and Cannabidiol (CBD, Cat. #104CBD) were purchased from BOL Manufacturing, Israel. Capsazepine (Cat. #0464) and Thapsigargin (Cat. #1138) were purchased from Tocris Bioscience, UK. Capsaicin (Cat. #M2028) was purchased from Sigma Aldrich. Camphor (Cat. #C352), Menthol (Cat. #M2772) and Carvacrol (Cat. #282197) were purchased from Sigma Aldrich.

### Cell Lines

The cancer cell lines MCF7, MDA-MB-231, MDA-MB-468, T47D, H1975, Panc1, BxPc3, Hs578T, B16, and LS174t and the monocyte/macrophage RAW 264.7 cell line were purchased from the American Type Culture Collection (ATCC). MCF7, MDA-MB-231, MDA-MB-468, Panc1, B16 and RAW 264.7 cells were maintained in Dulbecco's Modified Eagle Medium (DMEM, Biological Industries), T47D, H1975, and BxPc3 were maintained in RPMI 1640 Medium (Biological Industries), and LS174t cells were maintained in Minimum Essential Medium Eagle (Biological Industries). All media were supplemented with 10% Fetal bovine serum (FBS), 1% penicillin and streptomycin (Biological Industries) at 37° C. in a humidified atmosphere containing 5% CO₂.

### Cytotoxic Assays

Cells were plated at 4×10³/100 μl (MCF7, MDA-MB-468, H1975, Panc1, BxPc3, Hs578T, B16 and LS174t) or 8×10³/100 μl (MDA-MB-231, T47D) in 96-well plates in the corresponding medium and allowed to attach overnight. Then the medium was removed, the cells were washed twice with Phosphate Buffer Saline (PBS, Biological Industries), and a fresh, serum-free medium containing penicillin and streptomycin and the treatments/vehicle was added. Cell viability was assessed at 24 hours by using the Resazurin dye (Enco) as per manufacturer's instructions. The optical densities were quantified using a multi-well spectrophotometer. Results were calculated as the percentage of control cultures.

### Nitric Oxide (NO) Secretion

NO secretion was determined by measuring the amount of nitrite, using a Griess reagent (Promega) according to manufacturer's protocol. Briefly, 50 μl of supernatant was added to 96-well plate, followed by 50 μl sulphanilamide and 50 μl N−1-napthylethylenediamine dihydrochloride (NED). The optical densities were quantified using a multi-well spectrophotometer, and nitrite concentrations were estimated using a standard nitrite curve. Results were calculated as the percentage of control cultures.

### Results

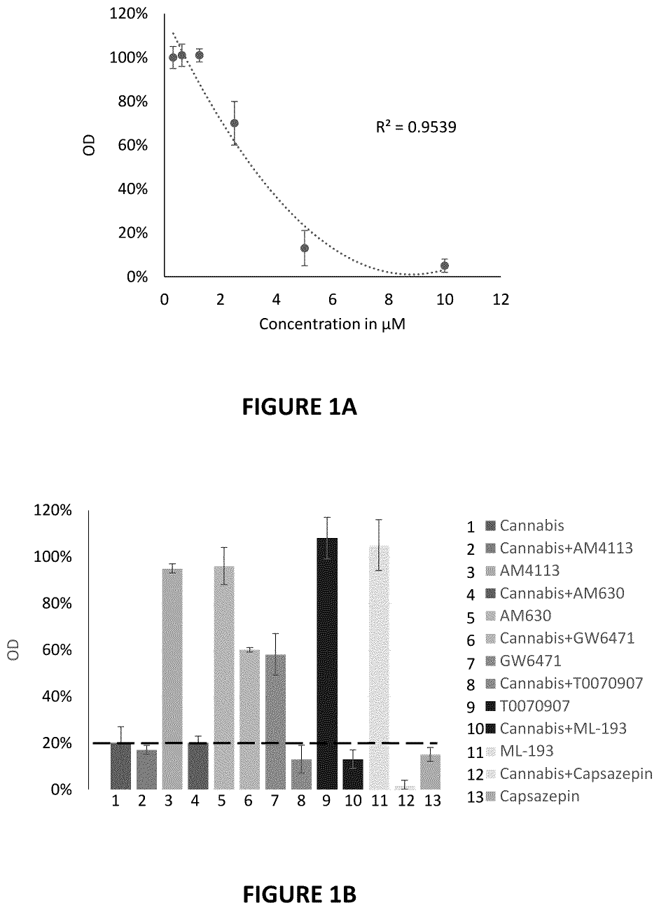

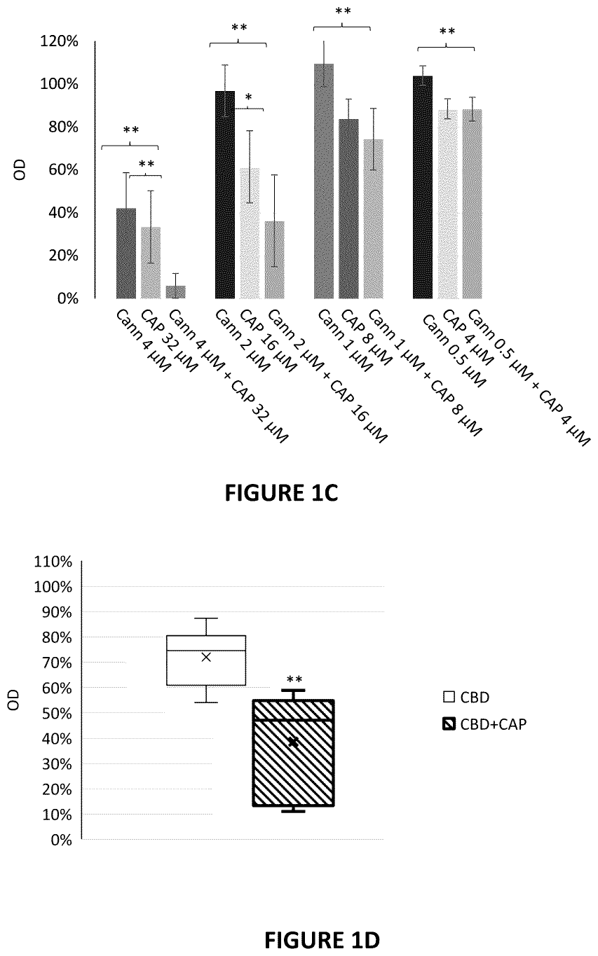

In this study the inventors tested the anti-tumorigenic effect of cannabis extract, which was enriched with cannabidiol (CBD) up to the level of 70% (volume/volume). The inventors exposed the triple negative breast cancer cell line MDA-MB-231 to various doses of said cannabis extract for 24 hours. The inventors then used the resazurin assay that quantifies the number of live cells as a measure for cytotoxicity. The result confirmed that CBD-enriched cannabis has a dose-dependent negative effect on cell survival (FIG. 1A), which was in-line with the current knowledge on the anti-cancer activity of CBD in-vitro. Nevertheless, when considering the dose requirement and the poor bioavailability of CBD, it was questionable whether a pronounced effect can be achieved in vivo. Thus, the inventors concluded that the naturally occurring CBD cannot serve as a stand-alone chemotherapy and sought to look for other agents that would further potentiate or enhance the anti-tumor activity of CBD-enriched cannabis.

CBD has a very low affinity for the major endocannabinoid receptors CB1 and CB2, and moreover, can act as an antagonist/inverse agonist at certain concentrations below the inhibition constant (Ki). To this end, the inventors exposed MDA-MB-231 cells to either CBD-enriched cannabis, a panel of synthetic molecules that block the ECS receptors and to combinations thereof (FIG. 1B). The data showed that while the vast majority of the ECS receptors antagonists tested (see Table 1 for details) either impaired cannabis activity or did not affect it, the TRPV1 antagonist capsazepine, had in fact enhanced the cytotoxic effect of cannabis, leading to a marked reduction in cell survival (31±10% survival for cannabis alone, 4±2% survival for cannabis+capsazepine, p. value <0.00001).

TABLE 1
List of antagonists used in this study and their respective targets.

| Antagonist name | Targeted receptor |
|---|---|
| AM4113 | CB1 |
| AM630 | CB2 |
| GW6471 | PPARα |
| T0070907 | PPARγ |
| ML-193 | GPR55 |
| Capsazepine | TRPV1 |

To have a better insight on the dose-response relationships of cannabis and capsazepine, the inventors then exposed MDA-MB-231 cells to diluted extract that contained a final concentration of 4 μM CBD, in the presence or absence of capsazepine. Dose response analysis showed consistently superior efficacy of the combination of cannabis and capsazepine over cannabis alone (p. value <0.0005), and over capsazepine alone (at the 2 highest doses tested; p. value <0.05 and 0.05, respectively, FIG. 1C).

Presumed synergistic effects between the active components of cannabis, collectively known as the entourage effect, suggest that the effectiveness of pure CBD is inferior to that of cannabis. To test whether the cytotoxic activity was related to CBD and not to other components of cannabis or to the entourage effect, the inventors exposed MDA-MB-231 cells to pure CBD, with or without capsazepine. The results showed that 2.5 μM CBD had a mild-to-moderate effect on cell survival (72±13%) while co-treatment with capsazepine significantly increased cancer cell death (39±21%, p. value <0.005, FIG. 1D). Therefore, the inventors concluded that pure CBD and capsazepine have a positive drug-drug interaction.

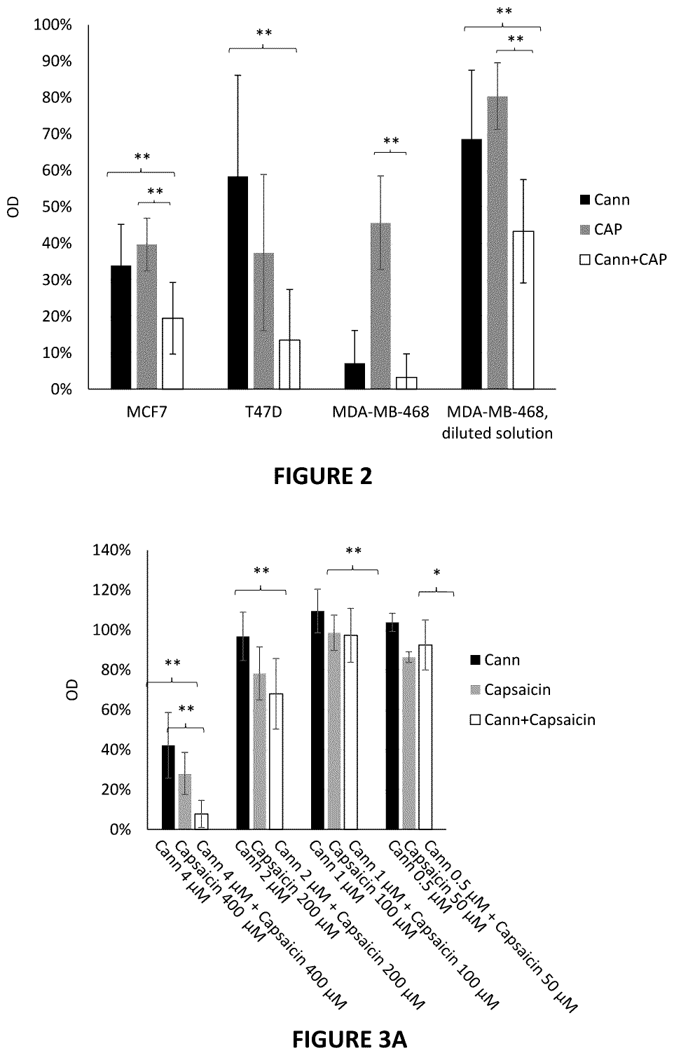

To investigate whether the positive interactions of cannabis/CBD and capsazepine is common to other breast cancer types, the inventors tested additional cell lines including MCF7 cells—derived from breast adenocarcinoma, MDA-MB-468 cells—derived from pleural effusion of triple negative metastatic adenocarcinoma, and T47D—derived from pleural effusion of a ductal carcinoma. MCF7 and T47D cells showed a moderate response to cannabis and capsazepine alone (cell survival of 34±11% and 58±28% for cannabis alone, and 40±7% and 37±21% for capsazepine alone, respectively) in comparison to the markedly enhanced effect of their combination (19±10% and 13±14%, respectively; FIG. 2). The effect was statistically significant in comparison to cannabis alone for both cell types (p. value <0.005), and significantly enhanced in comparison to capsazepine alone for MCF7 cells (p. value <0.0005). The cytotoxic effect of cannabis on MDA-MB-468 cells was highly pronounced and effective (7±9%), and the addition of capsazepine reduced cell survival to a staggering low level (3±6%; FIG. 2). To realize the full effect of the combination in comparison to each of the materials alone, the inventors further diluted both materials 1:2 and examined the resulting activity, as described. Cannabis containing 2 μM CBD had a mild-to-moderate effect on cell survival (69±19%), whereas 16 μM capsazepine had a very little effect (80±9%; FIG. 2). However, combination of the diluted compounds resulted with a statistically significant reduction of cell survival (43±14%; with p. value <0.005). These findings suggest that the elevated toxicity of cannabis/capsazepine combination is suitable for the treatment of cancer, such as breast cancer cell lines, as exemplified herein in cell derived from triple negative tumors.

Capsazepine is a known antagonist of the TRPV1 receptor and was extensively studied owing to its potential analgesic effect. Nevertheless, Phase I safety and tolerability studies on several synthetic TRPV1 antagonists have showed unforeseen adverse effects, including hyperthermia and impaired noxious heat sensation. Therefore, the inventors examined whether capsazepine can be substituted with natural compounds that are derived from medical herbs and are known from the literature to interact with the TRPV1 receptor. Importantly, the nature of interaction (agonism or antagonism) was not considered, as TRPV1 activation is followed by desensitization of the receptor. Thus, the inventors focused on the natural ligand of TRPV1 capsaicin, a capsaicinoid derived from Capsicum, and on the newly discovered ligand Thapsigargin, a sesquiterpene lactone derived from the plant Thapsia garganica, which exhibits both direct and indirect interactions with TRPV1.

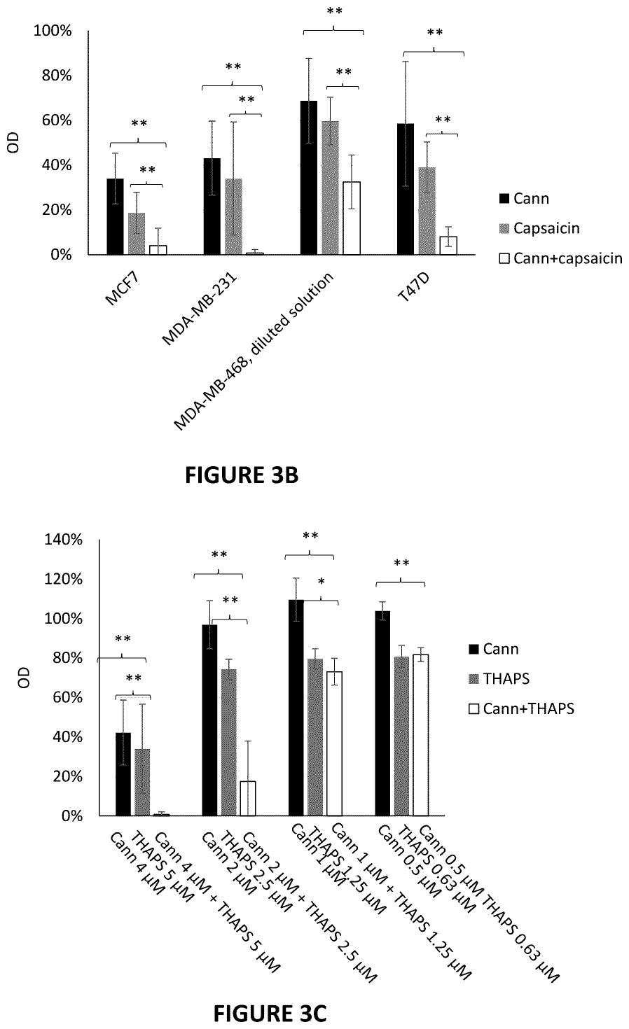

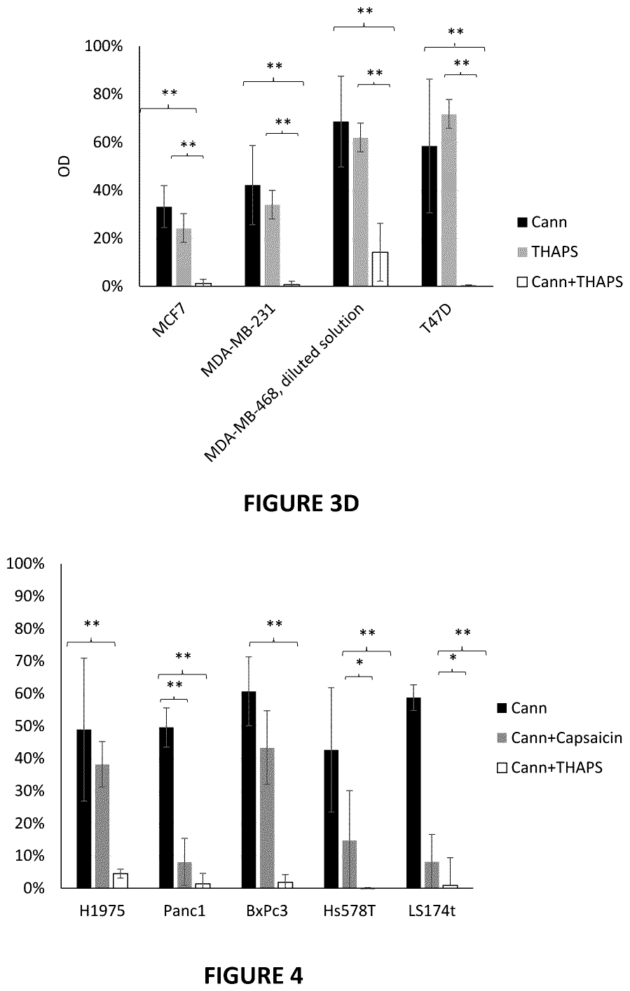

Treatment of MDA-MB-231 with cannabis extract containing various concentrations of CBD (0.5-4 μM), and/or various concentrations of capsaicin (50-400 μM) showed that the combined effect was significantly stronger than that of cannabis, especially at high doses (p. value ranges between 10 e⁻⁸ and 0.04, FIG. 3A). The effect of the combination was stronger than that of capsaicin alone at the highest dose tested (p. value <0.00005).

The inventors then tested the efficacy of the highest-dose combination on additional cell types. Co-exposure of MCF7, MDA-MB-231, and T47D cells to 400 μM capsaicin and cannabis containing 4 μM CBD had a significantly stronger cytotoxic effect than each of these compounds alone (4±8%, 1±2%, and 8±4%, for the combination versus 34±11%, 43±16%, and 58±28%, for cannabis, and 19±9%, 34±25%, and 39±11%, for capsaicin, respectively, p. value <0.005, FIG. 3A). The MDA-MB-468 cells, which strongly reacted to cannabis was exposed to 1:2 dilution of the original doses and demonstrated a similar improvement of the effect of the combination in comparison to cannabis and capsaicin alone (32±12% survival versus 69±19%, and 60±11%, respectively, p. value <0.0005, FIG. 3B).

Next, the inventors have tested the effect of combination of thapsigargin and cannabis on cell survival. It is known that thapsigargin has a strong apoptotic effect, which is caused by inhibition of the sarcoplasmic/endoplasmic reticulum Ca²⁺ ATPase (SERCA) pump. However, its high toxicity to normal cells hampers its usage as an anticancer drug. Thus, the inventors tested whether the combination of low dose thapsigargin and cannabis increases the cytotoxic effect of cannabis alone.

Treatment of MDA-MB-231 with cannabis extract containing various concentrations of CBD (0.5-4 μM), and/or various concentrations of thapsigargin (0.63-5 μM) showed that the combined effect was significantly stronger than that of cannabis for all doses tested (p. value ranges between 10 e⁻⁸ and 10 e-6, FIG. 3C). The effect of the combination was stronger than that of thapsigargin alone at the three highest doses tested (p. value ranges between 10 e⁻⁶ and 0.04), but not at the lowest dose tested.

The inventors then tested the efficacy of the highest-dose combination on additional cell types. Co-exposure of any one of MCF7, MDA-MB-231, and T47D cells to 5 μM thapsigargin and cannabis containing 4 μM CBD had a significantly enhanced cytotoxic effect than each of the compounds alone (2±2%, 1±1%, and 0.1±0.5% for the combination, versus 34±11%, 43±16%, and 58±28% for cannabis alone, and 24±6%, 34±23%, and 72±7% for thapsigargin alone, p. value <0.005, FIG. 3D). The MDA-MB-468 cells, which strongly reacted to cannabis were exposed to 1:2 dilution of the original doses and demonstrated a similar improvement of the effect of the combination compared to either cannabis alone or capsaicin alone (14±12% survival versus 69±19%, and 62±9%, respectively, p. value <0.0005, FIG. 3D).

Further, the inventors tested the effect of both combinations on cell lines derived from different origins, including non-small cell lung carcinoma (H1975), pancreas carcinoma (PANC1), pancreas adenocarcinoma (BxPC3), colon adenocarcinoma (LS174t), and an additional line of breast cancer which is classified as a carcinosarcoma (Hs578t). The results show that the cytotoxicity of cannabis that contained 4 μM CBD was moderate for all of the cell type tested (H1975, 49±22%; Panc1, 50±6%; BxPc3, 61±11%; Hs578T, 43±19%, LS174t, 59±4%). Co-exposure of the cells to cannabis and 5 μM thapsigargin resulted in marked and statistically significant reduction in cell survival (H1975, 4±1%; Panc1, 1±3%; BxPc3, 2±2%; Hs578T, 0±0.2%, LS174t, 1±0.2%, p. value <0.005). The effect of co-exposure of cannabis and capsaicin varied between the different cell line, with a significant reduction in cell survival in Panc1, Hs578T and LS174t cells (1±3%, p. value <0.0005, 15±15%, p. value <0.05, 8±8%, p. value <0.01, respectively) and a similar trend in H1975 and BxPc3 cells (H1975, 38±7%; BxPc3, 43±11, respectively). To conclude, the enhanced effect of cannabis and thapsigargin combination was seen across all malignant cell line tested, and the combination of cannabis and capsaicin was significantly toxic to most cell lines in the panel.

The poor bioavailability of CBD may hamper its effectiveness as an anti-inflammatory drug. To test whether antagonists of the ECS may potentiate the anti-inflammatory effect of CBD, the inventors used the RAW 264.7 macrophages as a model system for inflammation. Exposure of cells to bacterial lipopolysaccharide (LPS) initiates a signal transduction cascade that leads to increased production of nitrite oxide (NO), secretion of pro-inflammatory cytokines, and acquisition of enhanced bactericidal activity.

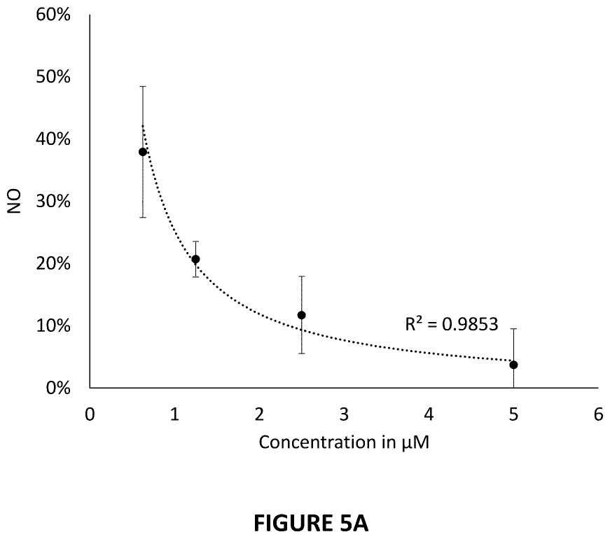

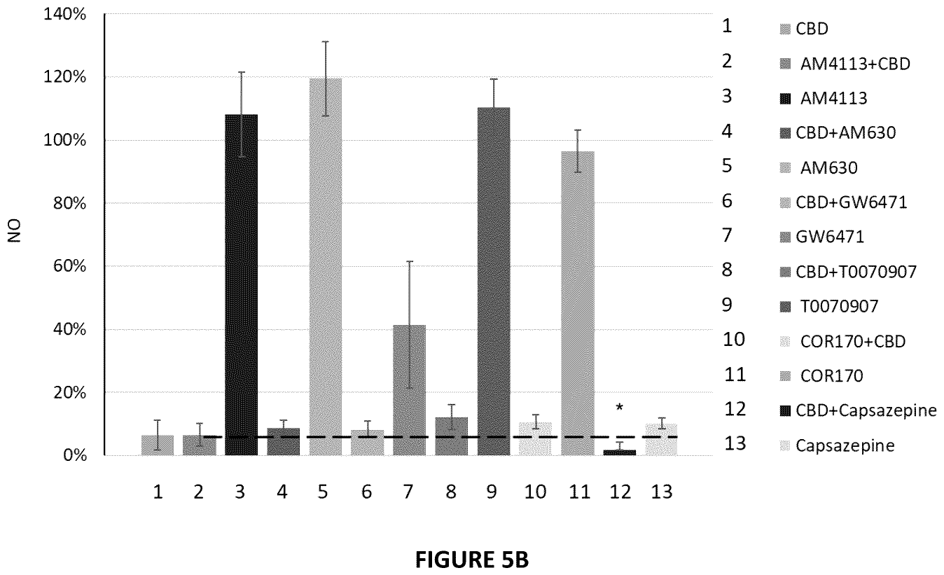

First, the inventors studied the response of LPS-activated RAW 264.7 cells to CBD. Cells were cultured with LPS, with/without CBD at 0.63-5 μM concentrations for 24 hrs. Supernatants were harvested for measuring NO by spectrophotometry using the Greiss reagent. The result showed that the exposure of the cells to CBD reduced NO secretion in a dose dependent manner (FIG. 5A). To test whether antagonists of the ECS can modify the anti-inflammatory activity of CBD, the inventors further exposed the cells to LPS, CBD, ECS antagonists (detailed in Table 2), and their combinations. Two distinct CB2 antagonists (AM630 and COR170) were tested, as CB2 is known to be involved in the regulation of inflammation. This experiment showed that at 2.5 μM, CBD reduced NO secretion to 6±5% of the one measured for control cells treated with vehicle alone (DMSO). Most of the antagonists used, except capsazepine and GW6471 (TRPV1 and PPARα antagonists, respectively), did not affect NO secretion. Moreover, the inventors observed that a combination of 2.5 μM CBD and 10 μM capsazepine significantly reduced NO secretion to 2±3%, p. value <0.05, suggesting that the combination has a more potent anti-inflammatory effect than CBD alone (FIG. 5B).

TABLE 2
List of antagonists used in this study and their respective targets.

| Antagonist name | Targeted receptor |
|---|---|
| AM4113 | CB1 |
| AM630, COR170 | CB2 |
| GW6471 | PPARα |
| T0070907 | PPARγ |
| Capsazepine | TRPV1 |

The potentiation of CBD is one of the crucial steps towards its development as an anti-cancer or anti-inflammatory drug, as it “suffers” from low bioavailability, therefore failing to obtain clinically meaningful outcomes upon using it as a monotherapy. The herein disclosed findings suggest that combination of CBD with either synthetic or naturally occurring TRPV1 ligands is highly toxic to cancer cells of many types and origins, and therefore is meaningful for treatment of cell proliferation related disease, e.g., cancer. In addition, the findings suggest that the described mechanism is also applicable for inflammation.

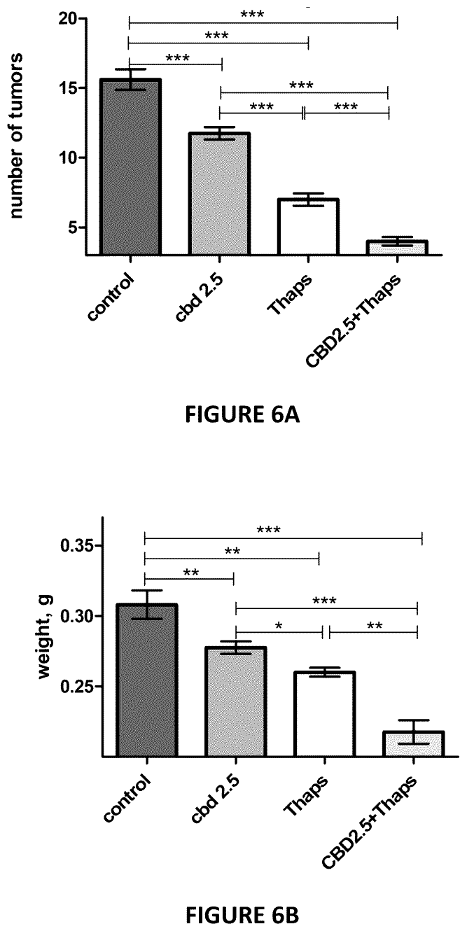

Further examining the synergistic anticancer activity of CBD with TRPV1 ligands, the inventors conducted an in-vivo study. Briefly, B16 melanoma cells were injected to C57B1 mice through tail vein, as a model of lung metastases. One (1) week after cell injection, the mice started the treatment with CBD, thapsigargin (TRPV1 ligand), or the combination of both. The combined treatment was significantly better than any one of the treatments separately (FIGS. 6A-6B).

In view of the above, the inventor further tested the clinical effect of other TRP ligands, other cannabinoids, and their combinations, in other indications, such as inflammation.

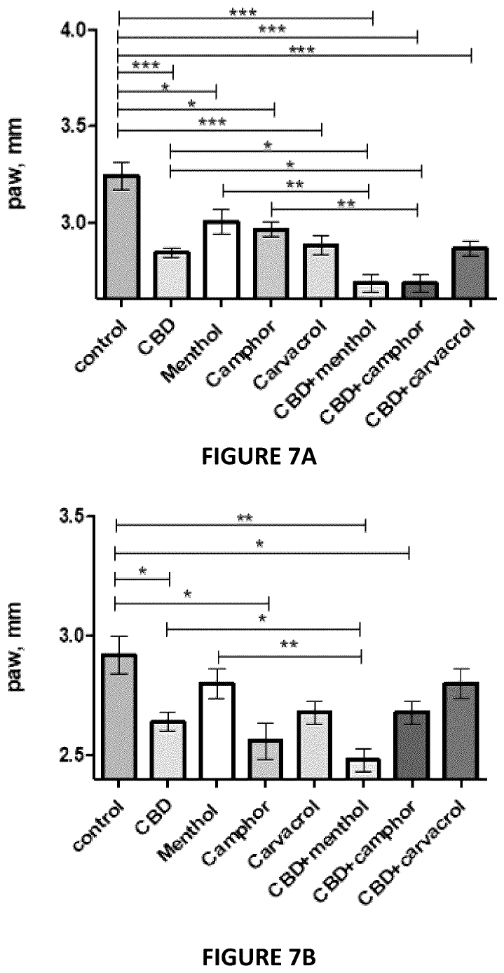

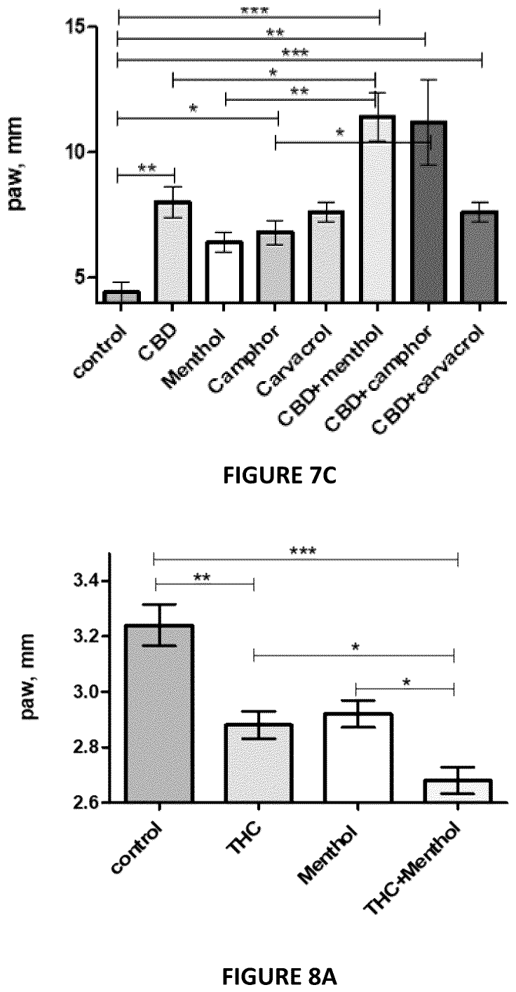

For instance, the effects of CBD on zymosan-induced paw swelling, pain, and tumor necrosis factor alpha (TNFα) levels were assayed in mice. The TRPA1 ligands: camphor, menthol, and carvacrol were tested, all of which are natural products known to be non-toxic in general. Combination of CBD with either camphor or menthol was found to have a therapeutically relevant effect, with menthol having a more significant effect (FIGS. 7A-7C).

Thereafter, the inventors sought to examine the effect of other cannabinoids. Specifically, the inventors replaced CBD with THC, and tested the compounds, and their combination, according to the same tests as described above (e.g., swelling, pain, and inflammation).

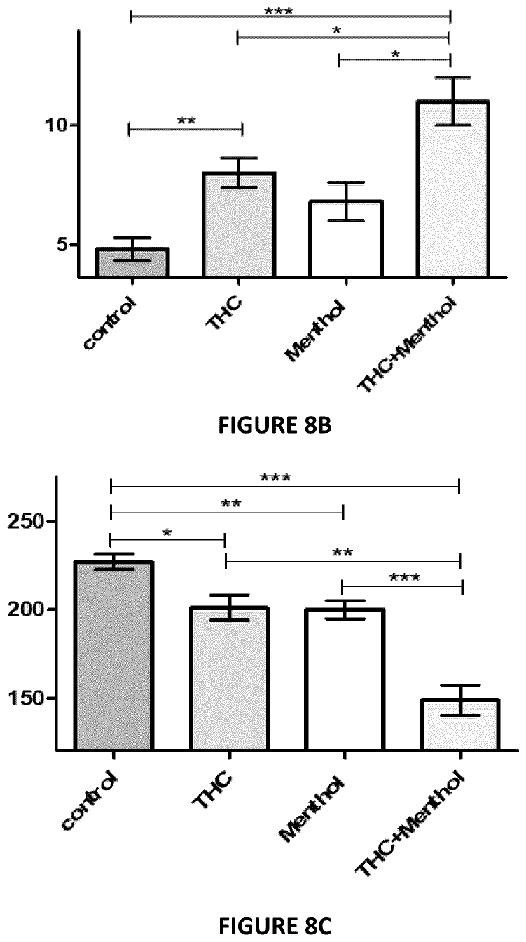

The results show that the combination of THC and menthol provided significantly improved results on the reduction of swelling, pain, and inflammation, compared to the application of each of the compounds individually (FIGS. 8A-8C, respectively).

Thus, the inventors suggest combining a cannabinoid and a TRP ligand as a synergistic composition for treating a cell proliferation-related disease. Such cell proliferation-related disease may include inflammation, cancer, as well as symptoms associated therewith, e.g., pain. This suggestion relies on the evidence presented herein showing that either CBD or THC, when combined with various TRP ligands, such as, but not limited to TRPV1 and TRPA1 ligands, provided a therapeutically relevant effect in the context of inflammation, cancer, and pain.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1.-18. (canceled)

19. A method for increasing or enhancing an activity of a cannabinoid in a subject in need thereof, comprising administering to said subject a pharmaceutical composition comprising a TRP ligand, wherein said activity is selected from the group consisting of: anti cell proliferation, anti-inflammation, analgesic, and any combination thereof.

20. A method for increasing or enhancing an activity of a TRP ligand in a subject in need thereof, comprising administering to said subject a pharmaceutical composition comprising a cannabinoid, wherein said activity is selected from the group consisting of: anti cell proliferation, anti-inflammation, analgesic, and any combination thereof.

21. The method of claim 19, wherein said administering is in a synergistically effective amount.

22. A method for ameliorating or treating a subject afflicted with a cell proliferation related disease, or a symptom associated therewith, the method comprising administering to said subject a therapeutically effective amount of a pharmaceutical composition comprising a cannabinoid and a TRP ligand, thereby ameliorating or treating the subject afflicted with the cell proliferation related disease.

23. The method of claim 22, wherein said administered cannabinoid and TRP ligand are at a molar ratio ranging from 1:1 (M:M) to 1:1,000 (M:M) in said pharmaceutical composition.

24. The method of claim 23, wherein said cannabinoid is selected from CBD or THC.

25. The method of claim 24, wherein said TRP is selected from TRPV1 or TRPA1.

26. The method of claim 25, wherein said TRPV1 ligand is selected from the group consisting of: capsazepine, capsaicin, thapsigargin, and any combination thereof.

27. The method of claim 25, wherein said TRPA1 ligand is selected from the group consisting of: menthol, camphor, carvacrol, and any combination thereof.

28. The method of claim 22, wherein said cannabinoid is present as a highly purified extract of Cannabis.

29. The method of claim 22, wherein said cannabinoid is synthetically produced.

30. The method of claim 22, wherein said cell proliferation related disease is selected from inflammation or cancer.

31. The method of claim 30, wherein said cancer is epithelial cancer.

32. The method of claim 31, wherein said epithelial cancer is cancer in a tissue selected from the group consisting of: breast, lung, pancreas, colon, and any combination thereof.

33. The method of claim 32, wherein said breast cancer comprises triple negative metastatic adenocarcinoma.

34. The method of claim 22, wherein said treating comprises: reducing the rate of cell proliferation, reducing the number of proliferating cells, reducing the survival rate of proliferating cells, reducing the levels of tumor necrosis factor alpha (TNFα), reducing pain associated with said cell proliferation related disease, or any combination thereof, in said subject
